(19) United States
(12) Patent Application Publication
(10) Pub. No.: US 2016/0120700 A1
(43) Pub. Date: May 5, 2016

(54) Intrastromal Corneal Reshaping Method and Apparatus for Correction of Refractive Errors Using Ultra-Short and Ultra-Intensive Laser Pulses

(71) Applicants: Taehee Han, Princeton, NJ (US); Szymon Suckewer, Princeton, NJ (US); Peter Hersh, Teaneck, NJ (US)

(72) Inventors: Taehee Han, Princeton, NJ (US); Szymon Suckewer, Princeton, NJ (US); Peter Hersh, Teaneck, NJ (US)

(21) Appl. No.: 14/874,407

(22) Filed: Oct. 3, 2015

Related U.S. Application Data
(60) Provisional application No. 62/074,365, filed on Nov. 3, 2014.

(51) Int. Cl. A61F 9/013; A61F 9/008

(52) U.S. Cl. CPC A61F 9/013; A61F 9/00814; A61F 9/00802; A61F 9/00804; A61F 2009/00872

(57) ABSTRACT

Ultra-short, ultra-intense laser pulses from a first laser beam are applied to a patient's cornea, creating a temporary micro-channel extending from the cornea surface to an end-point within it. Further ultra-short ultra-intense laser pulses from a second laser beam, are then delivered to the endpoint along with further pulses from the first beam, but delayed by a few nanoseconds. The micro-channel acts as a light-guide for these pulses. At the end point, they are focused to sufficient intensity to multiphoton ablate surrounding stromal tissue. With a few small entrance holes and without the lamellar flap necessary in LASIK procedures, the cornea is reshaped by rotating the direction of the laser beam. The vertical location of ablation is adjusted precisely using an applanator on the corneal surface. The multiphoton ablated tissue is ejected via the micro-channels, allowing the cornea surface to collapse after the procedure, changing its refractive power.

### CLAIM OF PRIORITY

This application claims priority to U.S. Provisional Application No. 62/074,365 filed on Nov. 3, 2014, the contents of which are hereby fully incorporated by reference.

### FIELD OF THE INVENTION

The invention relates to a methods and apparatus for ophthalmological treatment using femtosecond laser pulses to create ultra-strong electric fields that may be as strong as, or stronger than, the Coulomb fields containing valence electrons of the molecules and atoms of the cornea, thereby initiating multiphoton processes within the cornea, and more particularly to using ultra-intense laser pulses for intrastromal keratomileusis by multiphoton ablation for the correction of myopia, hyperopia, and astigmatism without the need for a lamellar LASIK flap.

### BACKGROUND OF THE INVENTION

Multiphoton processes may occur through the absorption of a significant number of photons by a single particle in a time shorter than the particle's relaxation times and/or by having the laser produce a local electric field sufficient strong to significantly or highly perturb the Coulomb force or potential of the particle. This nearly instantaneous absorption of energy by a particle, which may be a molecule, an atom, or an ion, causes that particle to break into its constituent parts. As discussed in detail later, this breakup may be a result of ionization of the valence electrons of the atom or molecule occasioned by either an absorption of sufficient photons to ionize one or more valance electrons, or by a combination of the laser pulse generating a local electric field having a strength sufficient to significantly, or highly, perturb the Coulomb field and multiphoton absorption of sufficient photons for one or more of the valance electrons to reach a tunneling threshold.

When such a process occurs within a solid, such as, but not limited to, the tissue in a cornea, the material within a region having a sufficiently high intensity to initiate a multiphoton process, disintegrates. This process is termed “multiphoton ablation”. A focused down, femtosecond laser beam can produce the necessary intensity of 10¹³ to 10¹⁵ W/cm², using only a few mille Joules of energy. The result is that femtosecond laser induced multiphoton ablation is highly localized and generates almost no heat or shock waves to surrounding tissue.

This is in marked contrast to thermal photo-ablation which requires a significantly lower intensity but significantly higher energy per pulse as only one photon at a time is absorbed by a particle. Thermal photo-ablation is typically performed using nanosecond lasers having pulse energies of tens of Joules, resulting in significant heating and shock to tissue surrounding the point of thermal photo-ablation.

The difference may best be understood using fluence rather than intensity. Fluence is simply the energy per unit area, typically expressed as Joules/cm² (J/cm²).

As an example, the deep ultraviolet, 10-20 nanosecond excimer laser pulses that are currently used in LASIK™ procedures have about 100 times higher threshold energy fluence (˜100 J/cm²) than the threshold energy fluence of a femtosecond laser pulse (˜1 J/cm2) for ablation of corneal tissue. There is, therefore, significantly less energy conversion to harmful mechanical effects such as heating or shock the surround tissue when using a multiphoton process with femtosecond pulses. Moreover, only multiphoton processes can create sufficiently narrow micro-channels in the cornea for the present invention.

This invention preferably uses ultra-short laser pulses, typically of 30 to 100 femtoseconds duration (1 femtosecond=1 fsec=1×10⁻¹⁵ sec), at a repetition of 1 kHz or higher, with a preferable intensity in the range of 10¹³-10¹⁵ W/cm². In a preferred embodiment, the laser output ultra-short beam may be split into 2 beams by a beam splitter in the laser output beam path. The femtosecond pulses of a first beam (the pre-pulses) create very narrow and long micro-channels in corneal tissue providing a high precision path for the femtosecond pulses of the second beam (the main pulses) with similar or higher intensity. When the pulses of the second beam reach the endpoint of the micro-channel, they reshape the intrastromal corneal tissue by multiphoton processes (“multiphoton ablation”) having had a minimal loss of energy during propagation through the channels.

### DESCRIPTION OF THE RELATED ART

With the development of compact ultra-short pulse lasers, the interaction between ultra-short laser pulse and tissue are being studied for applications in ophthalmology as ultra-short laser pulses, especially ultra-intensive pulses, can change the shape of a transparent eye tissue by multiphoton processes. These typically involve very localized, ultra-high electric fields. These electric fields may be comparable to or even larger than the Coulomb field in atoms and cause particles to disintegrate, but, because of their localization, cause minimal collateral damage to surrounding tissue. The multiphoton process produces very weak shock waves and heating effects in the tissue compared to longer pulses produced by lasers such as, but not limited to, the 0.5-10 nanosecond Alexandrite pulsed lasers, the 0.5-10 nanosecond Nd/YAG lasers or the 10-20 nanosecond excimer lasers. The pulses from these lasers may result in considerable trauma due to the much higher energy per pulse required for photo-ablation of corneal tissue. In fact, ultra-short laser pulses having very low energy and low intensity pulses, have already improved the quality of current laser eye surgery procedures. For instance, U.S. Pat. No. 5,993,438, issued to T. Juhasz et al. for “Intrastromal photorefractive keratectomy”, U.S. Pat. No. 6,110,116, issued to T. Juhasz for “Method for corneal laser surgery”, and U.S. Pat. No. 6,146,375, issued to T. Juhasz et al. for “Device and method for internal surface sclerostomy” teach about using low intensity femtosecond laser pulses for creation of a flap by photodisruption in conventional LASIK procedure. T. Juhasz et al.'s research has led to achieving much better precision and safety in cutting the flap than using a mechanical blade (microkeratome), while 5-20 nanosecond laser pulses are still being used for the corneal photo-ablation.

Though the methods to utilize a femtosecond laser for the flap creation has improved the quality of LASIK procedure, the creation of a flap may still cause damage on the surface layers of the cornea, corneal epithelium and Bowman's layer, leading to various flap complications such as flap striae, epithelial ingrowth, diffuse lamellar keratitis, flap tears, and corneal weakening resulting in ectasia.

Therefore, a new method that obviates the need for the creation of a flap by directly removing the stromal tissue have been sought, being expected to considerably improve corneal reshaping procedures and reduce the number of the complications. For example, in K. Koenig's US Patent Application 2004/0102765 A1 (May 27, 2004) “Method for minimal to non-invasive optical treatment of tissue of eye and for diagnosis thereof and device for carrying out said method” and M. Bendett et al.'s US Patent Application 2004/0243112 A1 (Dec. 2, 2004) “Apparatus and Method for Ophthalmologic Surgical Procedures using a Femtosecond fiber Laser”, methods to reshape the cornea using femtosecond lasers without creating a flap are described. However, a successful flapless corneal reshaping has not been achieved to date, primarily because no sufficiently high laser beam intensities have been applied that may allow multiphoton processes to take place in the cornea tissue. Because of this, no procedure has been able to avoid collateral damage to the surrounding tissue when laser pulses are applied to the ablation spots. Furthermore, a practical way to remove large amounts of ablated tissue from the cornea has not been achieved.

In U.S. Pat. No. 8,382,744, issued to S. Suckewer et al. for “Method and Device for Corneal Reshaping by Intrastromal Tissue Removal” (Feb. 26, 2013), a fundamentally new approach to cornea reshaping without creating a flap is described. In this invention, very high intensity ultra-short laser pulses are initially used to create a temporary micro-channel, which is oriented substantially normal to the optical axis of the cornea and extending to an end point located within the cornea. These ultra-short laser pulses are then delivered through the micro-channel to reshape corneal tissue by means of multiphoton processes, typically termed “multiphoton ablation”, in the vicinity of the end point of the micro-channel. The ablated tissue materials are ejected out of the cornea through the same micro-channel, which is used to deliver the laser pulses, or via a separate micro-channel. By supplying appropriate number of ultra-short laser pulses and by moving the point of ablation along the micro-channel, the cornea can be reshaped in a controlled fashion without creating a flap.

Various implementations of related procedures are known in the art, but fail to address all of the problems solved by the invention described herein. Various embodiments of this invention are illustrated in the accompanying drawings and will be described in more detail herein below.

### SUMMARY OF THE INVENTION

An inventive system and method of vision correction is disclosed.

In a preferred embodiment, the method may include removing stromal tissue of a mammalian cornea at a substantially constant distance from the surface of the cornea. The tissue removal is preferably accomplished using multiphoton ablation.

In a preferred embodiment, the void created within the cornea may be such that the upper surface of the void is a constant distance from a surface of the cornea as this may create more effective vision correction. This may, for instance, be accomplished by flattening the cornea before performing the multiphoton ablation.

A next step may include creating a temporary channel. This channel may extend from the side of the flattened cornea to the region in which the stromal tissue is to be removed. The channel may, for instance, be created using a first laser beam focused to have an intensity of between 10¹¹ and 10¹³ W/cm². This intensity may be sufficient to produce non-linear, self-focusing and plasma filament propagation in water and transparent solids such as stromal tissue. This filament propagation may result in micro-channels having diameters of 100 μm or less and lengths of 5 mm and more.

The temporary channel may then be used as an optical guide to direct a second, more intense laser beam that may be focused down to an intensity of at least 10¹³ W/cm² in a focal region. This intensity is sufficient to initiate multiphoton processes, resulting in multiphoton ablation in a vicinity of the focal region.

Once ablation is complete, the cornea may be un-flattened allowing the void created by removal of stromal to initially assume a curved shape with an upper surface being a constant distance from the anterior surface of the cornea. Once the void collapses, the shape of the cornea may then assume the desired shape providing the necessary change in refractive power to correct the vision defect.

As discussed in detail below, the method of the present invention may be used to correct myopia, astigmatism and hyperopia, which are the three most common human vision defects. In a preferred embodiment of the present invention, the laser beams may both originate from a common laser that preferably produces pulses having a pulse duration in a range of 10-500 femtoseconds. A pulse from the laser may, for instance, be split into two beams, the first beam, aka beam 1, being a lower intensity pre-pulse for producing the temporary channel, while the second beam, aka beam 2, may be a higher intensity pulse for producing multiphoton ablation. The higher intensity, main pulse may be delayed by 0.1-100 nanoseconds with respect to the lower intensity pulse from beam 1, allowing time for the micro-channel to be extended before the higher intensity pulse from beam 2 arrives.

The high intensity pulses from beam 2 may be temporarily blocked by a shutter until the having micro-channel has reached a point at which multiphoton ablation is required. Both pulses may then be delivered to a common focus so that the temporary micro-channel can continue to be extended, helping guide the laser further into the cornea.

The common laser may be a pulsed laser having a pulse duration in a range 10 to 500 femtoseconds. In a preferred embodiment, the pulsed laser may, for instance, be a laser such as, but not limited to, a Ti-Sapphire laser having a wavelength in a range of 750 to 850 nm, a pulse duration in a range of 30-100 femtoseconds, and has a repetition rate in a range of 0.1 to 10 kHz. In an alternative embodiment, the repetition rate may be in a range of 0.1 to 100 kHz.

Therefore, the present invention succeeds in conferring the following, and others not mentioned, desirable and useful benefits and objectives.

It is an object of the present invention to provide a method of minimally invasive treatment of vision defects.

Another object of the present invention is to provide a rapid and precise treatment of common vision defects resulting from an incorrect refractive power of the cornea.

### BRIEF DESCRIPTION OF THE DRAWINGS

FIG. 1 A shows a schematic cross sectional side view of a mammalian cornea.

FIG. 1 B shows a schematic cross sectional side view of a mammalian cornea having a surface flattened using an applanator.

FIG. 1 C shows a schematic cross sectional side view of a mammalian cornea having a region of stromal tissue removed while flattened.

FIG. 1 D shows a schematic cross sectional side view of a mammalian cornea having had a region of stromal tissue removed while flattened now un-flattened.

FIG. 1 E shows a schematic cross sectional side view of a mammalian cornea having had a region of stromal tissue removed while flattened with the void now collapsed.

FIG. 2 shows a schematic view of a preferred embodiment of the optics that may be used in implementing the present invention.

FIG. 3 A shows a schematic cross-sectional view of forming a micro-channel in accordance with a preferred embodiment of the present invention.

FIG. 3 B shows a schematic cross-section view of multiphoton ablation of a region of stromal tissue in accordance with a preferred embodiment of the present invention.

FIG. 4 shows a schematic view of a further preferred embodiment of the optics that may be used in implementing the present invention.

FIG. 5 shows a schematic view of yet a further preferred embodiment of the optics that may be used in implementing the present invention.

FIG. 6 shows a schematic plan view of multiphoton ablating a region of stromal tissue in accordance with a preferred embodiment of the present invention.

FIG. 7 shows a schematic plan view of multiphoton ablating a region of stromal tissue in accordance with a further preferred embodiment of the present invention.

FIG. 8 shows a schematic plan view of multiphoton ablating a region of stromal tissue in accordance with yet a further preferred embodiment of the present invention.

FIG. 9 A shows a diagrammatic drawing of a valance electron contained within a Coulomb potential being ionized via a multiphoton process.

FIG. 9 B shows a diagrammatic drawing of a valance electron contained within a significantly perturbed Coulomb potential being ionized via a multiphoton process that includes a degree of tunneling.

FIG. 9 C shows a diagrammatic drawing of a valance electron contained within a highly perturbed Coulomb potential being ionized via tunneling from a ground state.

### DESCRIPTION OF THE PREFERRED EMBODIMENTS

The preferred embodiments of the present invention will now be described with reference to the drawings. Identical elements in the various figures are, as far as possible, identified with the same reference numerals.

Reference will now be made in detail to embodiments of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

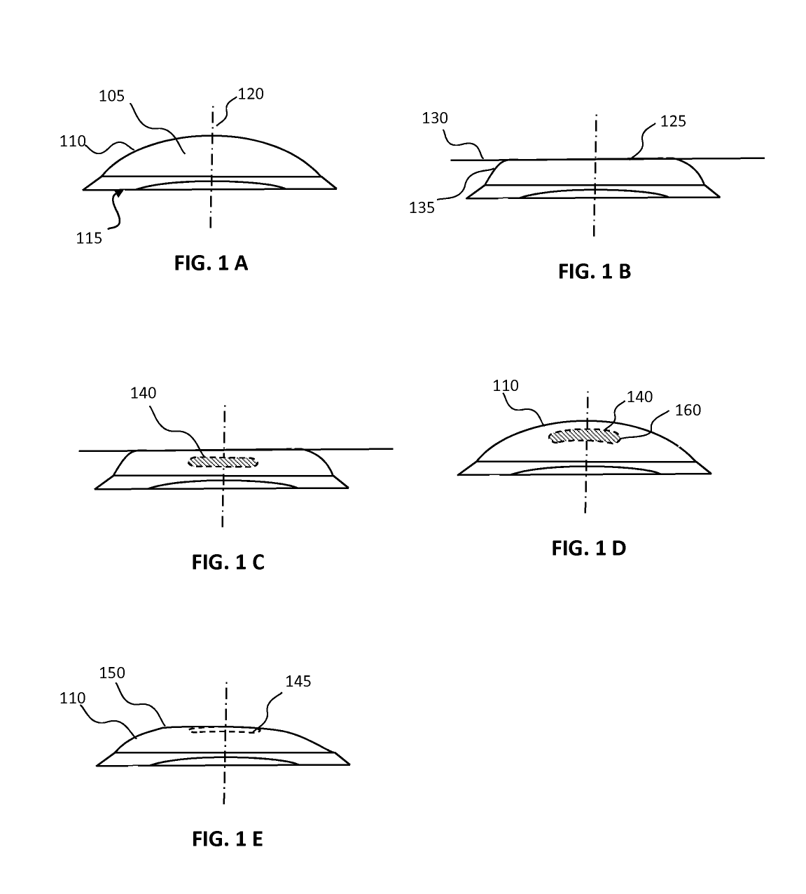

FIG. 1 A shows a schematic cross sectional side view of a mammalian cornea. This cross-section of a mammalian eye 115 shows how the stromal tissue 105 creates a curved surface of the cornea 110. This curvature may be rotationally symmetric about an optical axis 120 of an eye. The curvature of the cornea may play a significant role in determining the refractive power of the eye. Ideally the refractive power of the eye's lens and cornea, acting together, focus light onto the retina. Many defects of vision are the result of either too much or two little refractive power. When the eye has too much refractive power, light from distant objects may be focused in front of the retina. This is termed myopia and may be corrected by reducing the curvature of the cornea. When the eye has too little refractive power, light from distant objects may be focused behind the retina. This condition is called hyperopia and may be corrected by increasing the refractive power of the cornea by increasing the curvature of the surface of the cornea.

Another common vision defect is astigmatism. This may be a result of the refractive power of the eye being rotationally asymmetric. This problem may be corrected by shaping the surface of the cornea into the form of a sphero-cylindrical lens.

FIG. 1 B shows a schematic cross-sectional side view of a mammalian cornea having a surface flattened using an applanator 130. The applanator 130 may be a flat surface, such as, but not limited to, an optically flat microscope slide cover. The applanator 130 is preferably under precision control, particularly in a vertical direction, as the degree of flattening can help locate the area being multiphoton ablated more precisely. In a preferred embodiment, the applanator may be controlled by a micrometer with a micrometer thread capable of adjusting position to an accuracy of at least +/−10 μm, and with suitable equipment as little as +/−1 μm. Other position controllers of similar positioning resolution may also or instead be used including, but not limited to, an inch worm motion controller, a piezo-electric motion controller or a geared down stepper motor or some combination thereof.

FIG. 1 C shows a schematic cross sectional side view of a mammalian cornea having had a region of stromal tissue removed while flattened.

FIG. 1 D shows a schematic cross sectional side view of a mammalian cornea having had a region of stromal tissue removed while flattened now un-flattened. The result may be that the void 140 is now transformed from an essentially flat disk or thin spherical lens shape to a curved surface that may be rotationally symmetrical about the optical axis of the eye and that may have an upper surface located at a constant distance from the anterior surface 110 of the cornea

FIG. 1 E shows a schematic cross-sectional side view of a mammalian cornea having had a region of stromal tissue removed while flattened with the void now collapsed. The surface of the cornea 110 may now be transformed into having a flattened curvature 150, and therefore less refractive power. Such a reduction in curvature may be a correction for myopia.

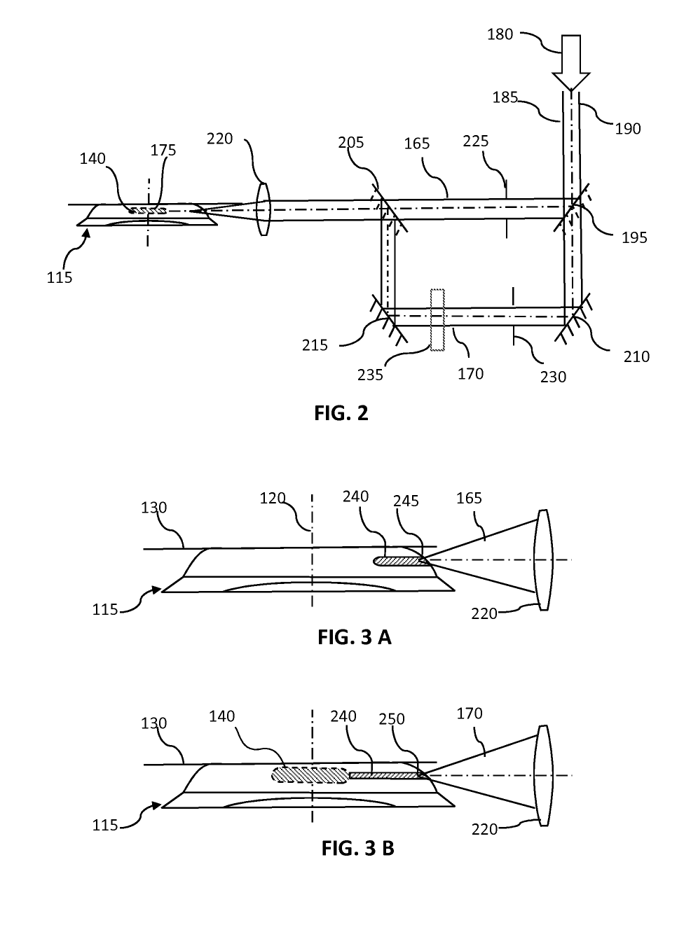

FIG. 2 shows a schematic view of a preferred embodiment of the optics that may be used in implementing the present invention.

As shown schematically in FIG. 2, a common laser 180 may be used to direct electromagnetic radiation towards a mammalian eye 115 for the purpose of removing stromal tissue by the process of multiphoton ablation 140.

Radiation from the common laser 180 may be split into a first laser beam 165 and a second laser beam 170 using a beam splitter 195. Beam splitters are well-known in the art and may be designed and manufactured to vary the amount of light that may go into each of the resultant output beams. Typical beam splitters may partition the light in any one of a chosen ratio from 90/10 ratio to a 50/50 ratio.

The first laser beam 165 may pass through a first aperture 225 and a beam combiner 205 before being focused by a focusing lens 220 to a preselected location on or in the cornea. The pulses of the first laser beam 165 are focused at a point in the cornea to create a temporary micro-channel oriented substantially perpendicular to the optical axis of the cornea. The center of the focus region, where the focal size of the beam is at the minimum, is preferably located near the corneal surface in order to keep the diameter of the entrance hole to the micro-channel in a range of 10-20 μm. The entrance holes of the micro-channels may be the only damage to the corneal surface that occurs during the procedure. These holes may, however, be very small and only temporary, typically disappearing in 2-5 minutes after the procedure is complete.

The second laser beam 170 may be redirected by a first mirror 210 to pass through a second aperture 230 and a second laser beam 170. The second laser beam 170 may then be redirected by a second minor 215 onto the beam combiner 205, thereby becoming once again co-axial with the first laser beam 165. The second laser beam 170 may then be focused by the focusing lens 220 onto a preselected spot on or in the cornea.

The main pulses of the second laser beam 170 may initially be blocked by a shutter. When the micro-channel reaches the ablation region to be removed, the main pulses may be “turned on” by allowing them to pass through the shutter. The main pulses, aka second laser beam 170, may pass through a delay line so as to arrive at the focal point 0.1 to 100 nanoseconds behind the pre-pulse, aka the first laser beam 165. This may allow the main pulse to be focused onto the pre-generated plasma, thereby being efficiently delivered deep into the cornea. The amount of stromal tissue removed may be precisely controlled by adjusting the pulse energy and the exposure time of the second laser beam 170, i.e., effectively by the adjusting the pulse energy and the number of pulses delivered. The size of the focal spot of the second laser beam 170, aka beam 2, is preferably sufficiently smaller than the size of the focal spot of the first laser beam 165, aka beam 1, so as to not to adversely affect the channel entrance created by pulses from the first laser beam 165.

The focal spot to which a laser beam may be focused is typically assumed to be proportional to the focal length of the focusing length and inversely proportional to the diameter of the beam, i.e., a smaller focal spot—and therefore greater intensity—may be obtained by either shortening the focal length of the focusing length, or by increasing the diameter of the beam striking the focusing lens.

The first aperture 225 may, for instance, be used to narrow the width or diameter of first laser beam 165 so as to reduce its intensity at the focal spot while second aperture 230 may be used to allow a wider diameter beam through so that second laser beam 170 may have a higher intensity at the focal spot than first laser beam 165 even though they are focused with the same focusing lens 220.

In a preferred embodiment, the common laser 180 may be a pulsed laser 185 having a pulse duration in a range 10 to 500 femtoseconds. In a more preferred embodiment the common laser 180 may be a Ti-Sapphire laser 190. The Ti-Sapphire laser 190 may, for instance, be a femtosecond laser with relatively low pulse energy but, because of the extremely short pulse length, may be focused to an intensity that may be as high as 10¹⁵ W/cm² at the focal spot. Typical parameters of a Ti-Sapphire laser 190 may include a wavelength in a range of 750 to 850 nm, a pulse duration in a range of 30-100 femtoseconds, and a repetition rate in a range of 0.1 to 10 kHz. Such a laser may be used to produce intensity as high as 10¹⁵ W/cm² using only 1-5 mJ of energy.

FIG. 3 A shows a schematic cross sectional view of forming a micro-channel in accordance with a preferred embodiment of the present invention.

A first laser beam 165 may be focused by a focusing lens 220 to a first focal point 245. The first laser beam 165 may be a suitable fractional split of the beam from the common laser 180 that may further be apertured down and then focused to have an intensity in a range of between 10¹¹ and 10¹³ W/cm². This may be sufficient to initiate non-linear effects such as, but not limited to, self-focusing and plasma filament propagation in the water and transparent solids that constitute the bulk of the stromal tissue within the cornea. A result of the plasma filament propagation may be to create a temporary micro-channel 240 extending from a surface of cornea to a micro-channel end point located within the cornea.

The diameter of the micro-channel is preferably smaller than 100 μm, and its length may be 3 mm or even as long as 5 mm, or in an alternative embodiment, even greater than 5 mm. The location of the micro-channel 240 is preferably in a range of 140-150 μm beneath the anterior surface of the cornea. The orientation of the micro-channel is preferably perpendicular to the optical axis of an eye 120 and parallel to the under-surface of the applanator 130.

While the temporary micro-channel 240 is being created, the second laser beam 170 may be stopped by the shutter 235 (shown in FIG. 2).

FIG. 3 B shows a schematic cross-section view of multiphoton ablation of a region of stromal tissue in accordance with a preferred embodiment of the present invention.

The second laser beam 170 may be a sufficient fraction of the beam from the common laser 180 and may be apertured and focused to produce an intensity in the vicinity of the focal spot of between 10¹³ and 10¹⁵ W/cm². This intensity is sufficient to initiate a multiphoton process, resulting in multiphoton ablation of the stromal tissue in the vicinity of the focal spot.

A void 140 may be created by the multiphoton ablation. The vapor and any liquid produced by the multiphoton ablation in creating this void may be expelled via the temporary channel 240 by an ablation produced pressure increase within the void.

In a preferred embodiment, the pulse associated with the first laser beam 165 may arrive up to a 100 nsecs before the pulse associated with the second laser beam 170 although they are split from the same initial pulse produced by the common laser 180. This may be accomplished by the longer path taken by the second laser beam 170. An extra path of 1 ft. may result in a delay of approximately 1 nsec. The pulse from the first beam, aka beam 1, may, therefore, be described as the pre-pulse and the pulse from the second beam, aka beam 2, as the main pulse as it may be responsible for initiating the multiphoton processes responsible for multiphoton ablation.

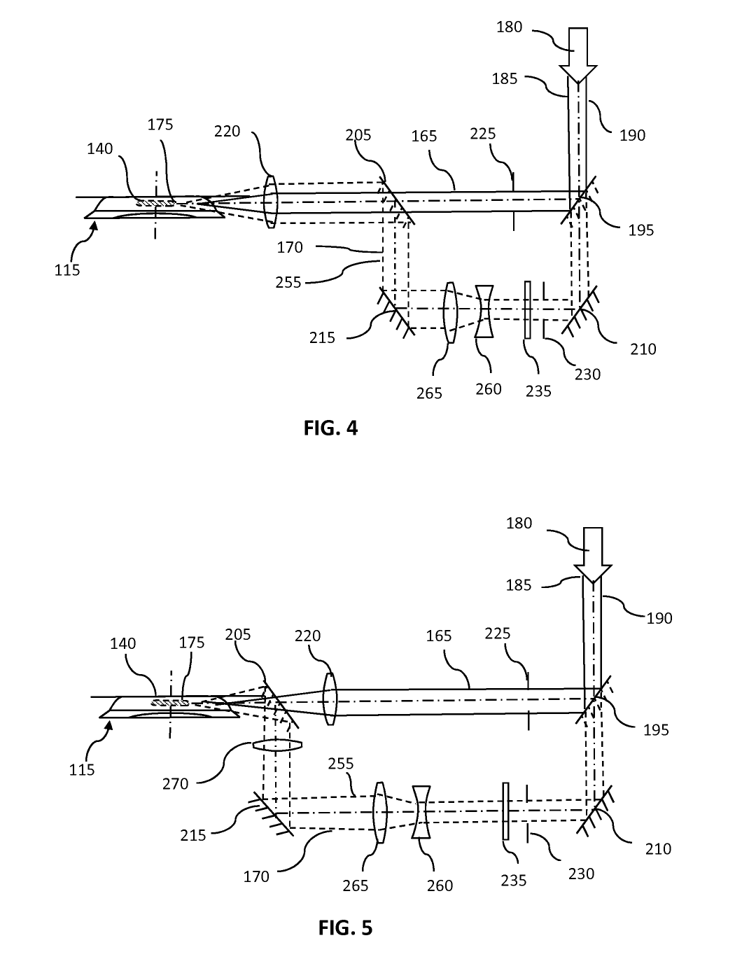

FIG. 4 shows a schematic view of a further preferred embodiment of the optics that may be used in implementing the present invention.

In the layout shown in FIG. 4, a beam expanding combination of a diverging lens 260 and a converging lens 265 may be added in the path of the second laser beam 170, aka beam 2. These optics are preferably placed between the second mirror 215 and the shutter 235. One of ordinary skill in the art will, however, appreciate that the beam expanding combination may be placed anywhere in the path of the second laser beam 170, aka beam 2, from the beam splitter 195 to the beam combiner 205. The effect of having the second laser beam, aka beam 2, expanded 255 may be to increase the intensity of the beam when focused by the focusing lens 220. The optics in the path of the first laser beam 165, aka beam 1, may be unchanged from the version shown in FIG. 2, i.e., the beam from the common laser 180 is split at the beam splitter 195 then proceeds through the first aperture 225 and the beam combiner 205.

The common laser 180 is preferably a pulsed laser 185 having a pulse duration in a range 10 to 500 femtoseconds and more preferably a Ti-Sapphire laser 190 with wavelength in a range of 750 to 850 nm, a pulse duration in a range of 30-100 femtoseconds, and has a repetition rate in a range of 0.1 to 10 kHz.

FIG. 5 shows a schematic view of yet a further preferred embodiment of the optics that may be used in implementing the present invention.

In the optical configuration shown in FIG. 5, the second laser beam 170, aka beam 2, may be both expanded and the expanded second laser beam 255 focused down using a second focusing lens 270, distinct from the focusing lens 220.

This may, for instance, be implemented by moving the focusing lens 220 from between the beam combiner 205 and eye 115 to be in the path of the first laser beam 165, aka beam 1, between the focusing lens 220 and the first aperture 225. The second focusing lens 270 may then be placed between the beam combiner 205 and the second minor 215. The beam expanding combination of the converging lens 265 and the diverging lens 260 is shown in FIG. 5 located between the second mirror 215 and the shutter 235. One of ordinary skill in the art will, however, appreciate that the beam expanding combination may be situated anywhere in the path of the first laser beam 165, aka beam 1, between the second focusing lens 270 and beam splitter 195. The shutter 235 and the second aperture 230 may similarly be placed anywhere in the path of the second laser beam 170, aka beam 2, between the diverging lens 260 and the beam splitter 195. Similarly the shutter 235 may be placed at any position between the beam combiner 205 and the beam splitter 195.

The common laser 180 may be a pulsed laser 185 having a pulse duration in a range 50 to 100 femtoseconds and is preferably a Ti-Sapphire laser 190 with wavelength in a range of 750 to 850 nm, a pulse duration in a range of 30-100 femtoseconds, and has a repetition rate in a range of 0.1 to 10 kHz.

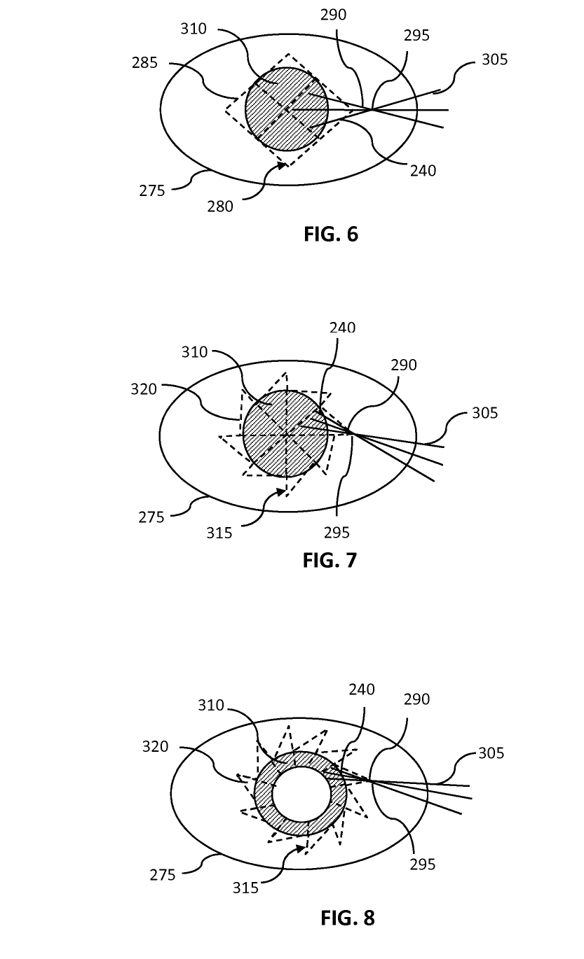

FIG. 6 shows a schematic plan view of multiphoton ablating a region of stromal tissue in accordance with a preferred embodiment of the present invention.

The arrangement shown in FIG. 6 is intended to correct for myopia, the condition in which the refractive power of the cornea is too great, resulting in distant objects being imaged ahead of the retina. The condition may be corrected by a suitable reduction in the refracting power of the cornea, which may be accomplished by reducing the curvature of the surface of the cornea. One method to achieve this may be to create a void that may be shaped in the form of a thin, converging spherical lens having an axis of rotation parallel to and coincident with an optical axis of the eye. This may be achieved by removing stromal tissue.

One arrangement to achieve this using the two-pulse technique described above, may be to divide the cornea into four quadrants 285 inside a bounding rectangle 285. A sector of the thin, converging spherical lens may then be created in each quadrant using only one entrance 290 per quadrant. The line of travel 305 of the laser pulses may be rotated around a center of rotation 295 that may be coincident with the entrance 290 of the temporary channel. In this manner the entire thin lens shaped void may be created using only four entrance holes. This may make the procedure minimally invasive by keeping the damage to the surface of the cornea as small as possible, thereby minimizing the chances of bacterial infection to the cornea during and after the procedure.

The thickness of the lens may be accurately controlled using a micrometer adjustable applanator to control the depth beneath the anterior surface of the cornea at which the multiphoton ablation occurs, as described above.

Once the applanator is removed, the lens may initially assume the shape of a curved, rotationally symmetric lens, having an upper curved surface that may be a constant distance from the anterior surface of the cornea. When this void collapses, the curvature of the surface of the cornea may assume a flatter shape resulting in a reduced refractive power of the cornea that may correct the original myopia.

FIG. 7 shows a schematic plan view of multiphoton ablating a region of stromal tissue in accordance with a further preferred embodiment of the present invention.

The configuration shown in FIG. 7 is intended to correct astigmatism. This is a condition in which light in a vertical plane is focused on the retina, light in a horizontal plane is focused either ahead of or behind the retina. To correct this defect, the cornea may need to be shaped in the form of a sphero-cylindrical lens having an optical axis parallel to and coincident with an optical axis of the eye. This may be done using multiphoton ablation by creating a void that is shaped, while flattened, in the form of a sphero-cylindrical lens having an optical axis parallel to and coincident with an optical axis of the eye that may essentially be a negative of a lens that may correct the astigmatism.

Because of the more complex shape to be formed, there may need to be more entrances 290 for the temporary channel. FIG. 7 shows a bounding polygon 315 divided into eight triangular region 320. The region to be removed by multiphoton ablation 310 in each triangular region 320 may be addressed by having the line of travel 305 of a laser pulse be rotated about the center of rotation 295 that is preferably coincident with an entrance 290. As before the height at which the ablation occurs may be adjusted using the vertical position of the applanator. The applanator may control all or part of the height adjustment.

When the applanator is removed, the void may transform into a more curved sphero-cylindrical lens shape having an upper surface that may be at a constant depth beneath the anterior surface of the cornea. When the void collapses the surface of the cornea may then assume a surface shape of a sphero-cylindrical lens, and may correct the astigmatism.

FIG. 8 shows a schematic plan view of multiphoton ablating a region of stromal tissue in accordance with yet a further preferred embodiment of the present invention.

The configuration shown in FIG. 8 is intended to correct hyperopia. This is a condition in which light from a distant object is focused beyond the retina because the cornea has too little refractive power. To correct this defect the refractive power of the cornea needs to be increased which may be done by increasing the curvature of the surface of the cornea. To achieve this using multiphoton ablation, for a least the central portion of the cornea, a void may need to be created that while the cornea is flattened, is in the form of a torus, i.e., a doughnut like, toroidal shape formed by rotating a circle about a central axis. The central axis of the torus is preferably coincident with the optical axis of the eye.

Because of the more complex shape to be formed, there may need to be more entrances 290 for the temporary channel. FIG. 8 shows a bounding polygon 315 divided into twelve triangular regions 320. The region to be removed by multiphoton ablation 310 in each triangular region 320 may be addressed by having the line of travel 305 of a laser pulse be rotated about the center of rotation 295 that is preferably coincident with an entrance 290. As before the height at which the ablation occurs may be adjusted using the vertical position of the applanator. The applanator may control all or part of the height adjustment.

When the applanator is removed, the void may transform into a toroidal shape having a slightly elliptical cross-section. When the void collapses the surface of the cornea may then fall into the toroidal trench. This may result in an increased curvature of the cornea in the vicinity of the optical axis of the eye, which may increase the curvature of the central portion of the cornea. This increased curvature may increase the refractive power of the cornea and may correct the hyperopia.

The focusing and expanding optical elements in the optical configurations above have all been described as lenses. As one of ordinary skill in the art will, however, appreciate suitably curved minors, or a combination of curved minors and lenses may be substituted for any or all of the focusing or expanding elements described above.

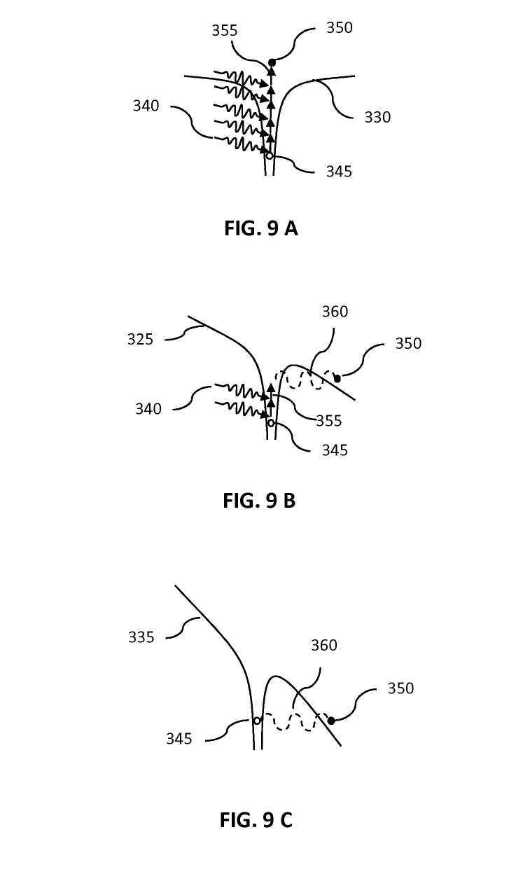

FIG. 9 A shows a diagrammatic drawing of a valance electron contained within a Coulomb potential being ionized via a multiphoton process. In the diagram of FIG. 9A, there is an unperturbed Coulomb field 330 constraining a trapped valence electron 345. The arrival of multiple photons 340 within a laser pulse in a sufficiently short time results in the packets of energy 355 deposited by each single photon being additively absorbed by the electron, allowing the electron to gain sufficient energy to escape the unperturbed Coulomb field 330 and become a free, ionized electron 350.

FIG. 9 B shows a diagrammatic drawing of a valance electron contained within a significantly perturbed Coulomb potential being ionized via a multiphoton process that includes a degree of tunneling.

The significantly perturbed Coulomb field 325 may be caused by the ultra-intense local electric field generated by the high intensity focused femtosecond laser pulse. As a result of this perturbation, the trapped valence electron 345 may only need to be raised to a significantly lower energy level by the absorption of packets of energy 355 deposited by a few photons 340 within the femtosecond laser pulse. Once raised to this energy level, the trapped valence electron 345 may then be able to follow a quantum tunneling path 360 to become a free, ionized electron 350.

FIG. 9 C shows a diagrammatic drawing of a valance electron contained within a highly perturbed Coulomb potential being ionized via tunneling from a ground state.

The highly perturbed Coulomb field 335 results in the trapped valence electron 345 having sufficient energy to follow a quantum tunneling path 360 and become a free, ionized electron 350.

The three mechanisms described may be characterized by the Keldish parameter. This is defined as

$$γ = {\frac{ω}{e} [ \frac{mcn {ɛ}_{0} {E}_{g}}{I} ]}^{\frac{1}{2}}$$

where ω is the laser frequency, I is the laser intensity at the focus, m and e are the reduced mass and charge of the electron, c is the velocity of light, n is the refractive index of the material, E_{g} is the band gap of the material and ε₀ is the permittivity of free space.

FIG. 9 A corresponds to a situation in which the Keldish parameter, γ, is greater than 1.5. For an 800 nm laser operating on water, which is the main constituent of cellular material, this is typically an intensity of 10¹³ W/cm² or lower.

FIG. 9 B corresponds to a situation in which the Keldish parameter, γ, is equal to 1.5. For an 800 nm laser operating on water, which is the main constituent of cellular material, this is typically an intensity in a range of 10¹³ W/cm² to 10¹⁴ W/cm².

FIG. 9 C corresponds to a situation in which the Keldish parameter, γ, is less than 1.5. For an 800 nm laser operating on water, which is the main constituent of cellular material, this is typically an intensity in a range of 10¹⁴ W/cm2 to 10¹⁵ W/cm².

The Keldish parameter is described in detail in publications such as, but not limited to, the article entitled “Atomic Physics with Super-High Intensity Lasers” by Protopapas et al. published in Rep. Prog. Phys. 60 (1997) 389-486, the contents of which are hereby incorporated in their entirety.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed:

1: A method of vision correction, comprising
removing stromal tissue at a substantially constant distance from a surface of a cornea using multiphoton ablation, said removing comprising:
flattening said surface of said cornea; and
while said surface of said cornea is flattened:
creating a temporary channel from a side of said flattened cornea, substantially parallel to the flattened surface of said cornea, and extending from said side to a predetermined location within said cornea, using a first laser beam focused to have an intensity in a range of 10¹¹ to 10¹³ W/cm²;
directing a second laser beam through said temporary channel to said predetermined location, said second laser beam being focused to have an intensity of at least 10¹³ W/cm² thereby creating a localized electric field having a strength sufficient to create a significantly perturbed Coulomb field of an atom or a molecule within a focal region of said second laser beam and hence initiate multiphoton and/or tunneling ionization of electrons, causing multiphoton ablation of said stromal tissue in said vicinity of said predetermined location; and
after said multiphoton ablation is complete, unflattening said surface of said cornea.

2: The method of claim 1 wherein both said first and said second laser beam emanate from a common laser.

3: The method of claim 2 wherein said common laser is a pulsed laser having a pulse duration in a range 30 to 100 femtoseconds.

4: The method of claim 3 wherein the pulses in said first laser beam arrive at a first focal point within the cornea in advance of said second laser arriving at a second focal point within the cornea by a time in a range of 0.1-100 nsec.

5: The method of claim 4 wherein said laser is a Ti-Sapphire laser with a wavelength in a range of 750 to 850 nm, a pulse duration in a range of 10-500 femtoseconds, and a repetition rate in a range of 0.1 to 10 kHz.

6: The method of claim 1 further comprising correcting for myopia, said method comprising:
ablating said stromal tissue to create a void that is shaped, while flattened, in the form of a thin, converging spherical lens having an axis of rotation parallel to and coincident with an optical axis of the eye.

7: The method of claim 1 further comprising correcting for hyperopia, said method comprising: ablating said stromal tissue to create a void that is shaped, while flattened, in the form of a toroid having an axis of rotation parallel to and coincident with an optical axis of the eye.

8: The method of claim 1 further comprising correcting for astigmatism, said method comprising: ablating said stromal tissue to create a void that is shaped, while flattened, in the form of a sphero-cylindrical lens having an optical axis parallel to and coincident with an optical axis of the eye.

9: The method of claim 1 wherein said flattening comprises applying an applanator to the surface of the cornea.

10: The method of claim 9 wherein said applanator has an optically flat, lower surface in touch with said surface of said cornea, and a vertical control device that positions said optically flat lower surface with a vertical precision of at least 10 μm.
